(19) United States
(12) Patent Application Publication
(10) Pub. No.: US 2025/0303072 A1
(43) Pub. Date: Oct. 2, 2025

(54) A SUBASSEMBLY OF A MEDICAMENT DELIVERY DEVICE

(71) Applicant: SHL Medical AG, Zug (CH)

(72) Inventor: Anders Boström, Nacka Strand (SE)

(21) Appl. No.: 18/865,844

(22) PCT Filed: May 8, 2023
(86) PCT No.: PCT/EP2023/062092

§ 371 Date: Nov. 14, 2024

(30) Foreign Application Priority Data
May 19, 2022 (EP) 22174284.4

(51) Int. Cl. A61M 5/32

(52) U.S. Cl. CPC A61M 5/3213; A61M 5/3204; A61M 2207/00

(57) ABSTRACT

The present disclosure generally relates to medicament delivery devices such as autoinjectors, and particularly concerns a subassembly for removing a needle shield.

### CROSS-REFERENCE TO RELATED APPLICATIONS

The present application is a U.S. National Phase Application pursuant to 35 U.S.C. § 371 of International Application No. PCT/EP2023/062092 filed May 8, 2023, which claims priority to EP patent application Ser. No. 22/174,284.4 filed May 19, 2022. The entire disclosure contents of these applications are herewith incorporated by reference into the present application.

### TECHNICAL FIELD

The present disclosure generally relates to medicament delivery devices such as autoinjectors, and particularly concerns a subassembly for removing a needle shield.

### BACKGROUND

A number of medical conditions require injections. These days, a number of different injection devices exist, including various types of pen injectors, autoinjectors and on-body devices. Although many of these devices have enabled major improvements in the management of a number of medical conditions, various limitations do still exist in the current technology. Not least amongst these are the difficulties faced by patients that require frequent injections and by patients that need to inject particularly viscous drugs. In considering these problems, the applicant has appreciated that various developments could be made to help improve the medicament delivery devices on the market today, for example concerning removal of a needle shield of the medicament delivery devices, which are set out in more detail below.

### SUMMARY

An object of the present disclosure is to provide a subassembly for a medicament delivery device which solves, or at least mitigates problems of the prior art.

According to a first aspect of the present disclosure, there is provided a subassembly of a medicament delivery device, the subassembly comprising: a syringe housing having a distal syringe housing part and a proximal syringe housing part, the distal syringe housing part comprising a flexible arm extending towards the proximal syringe housing part, the flexible arm comprising a protrusion configured to engage with a proximally facing surface of the proximal syringe housing part, a shield member configured to cover a medicament delivery member, the medicament delivery member being attached to a proximal end of a medicament container accommodated in the syringe housing; and a cap configured to cover the shield member, the cap comprising at least one flexible arm extending distally towards the syringe housing, the cap further comprising at least one flexible rib located between the flexible arm of the distal syringe housing part and the proximal syringe housing part to prevent engagement between the protrusion of the flexible arm of the distal housing part and the proximally facing surface of the proximal syringe housing part, wherein the at least one flexible arm of the cap comprises a radial protrusion configured to engage with a distally facing surface of the proximal syringe housing part, wherein when the cap is disassembled from the syringe housing by moving the cap proximally relative the syringe housing, the engagement between the radial protrusion of the at least one flexible arm of the cap and the distally facing surface of the proximal syringe housing part causes the proximal syringe housing part to move proximally relative the distal syringe housing part.

Embodiments of the present disclosure advantageously provides for removal of the shield member by pulling off the cap. The syringe housing comprises two separable parts, the distal syringe housing part and the proximal syringe housing part. The proximal syringe housing part is adapted to push on the shield member so that the shield member is moved proximally with respect to the needle so that the needle is exposed once the cap is removed. This herein disclosed subassembly enables a shield member removal using components and parts that can be made in more cost-efficient ways, for example by avoiding the use of stainless steel.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the components thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which under use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, “circumferentially” refer to a circumference or a circumferential direction 301 relative to an axis 102, typically a central axis extending in the direction of the longest extension of the device and/or component. Similarly, “radial” or “radially” refer to a direction 302 extending radially relative to the axis, and “rotation”, “rotational” and “rotationally” refer to rotation relative to the axis.

According to one embodiment, the shield member may comprise a distally facing surface configured to engage with a proximally facing surface of the proximal syringe housing part, whereby the shield member is configured to move in the axial direction to expose the medicament delivery member. The motion of the shield member is sufficient to completely remove the shield member from the needle.

According to one embodiment, wherein the protrusion of the at least one flexible arm of the cap may extend radially inwards and comprises a proximally facing surface configured to engage with the distally facing surface of the proximal syringe housing part. This provides for a simple yet robust engagement between the cap and the proximal syringe housing part.

According to one embodiment, the proximal housing part may comprise a groove or hole, wherein the distally facing surface is a side wall of the groove or hole.

According to one embodiment, the rib is a first rib, and wherein the cap may comprise a second rib opposite the first rib with respect to the longitudinal axis.

According to one embodiment, the flexible arm may be one of a first pair of flexible arms of the cap, the cap may further comprise a second pair of flexible arms opposite the first pair of flexible arms with respect to the longitudinal axis. A symmetric arrangement of two pairs of flexible arms provides for a more rigid attachment between the cap and the proximal syringe housing part, and for a more reliable motion of the proximal syringe housing part as the cap is pulled off and thereby a more reliable removal of the shield member.

According to one embodiment, the protrusion of the flexible arm of the distal syringe housing part may extend radially inwards.

According to one embodiment, the proximal syringe housing part may comprise a groove radially inside the flexible arm of the distal syringe housing part for receiving the rib. This groove is advantageously adapted to guide the rib in under the flexible arm of the distal syringe housing part when the cap is being assembled to the syringe housing.

According to one embodiment, the distal end of the distal syringe housing part may be configured to abut against a flange on a distal end of the medicament container. This advantageously ensures that the distal housing part does not move in the distal direction with respect to the medical container when the cap is assembled to the syringe housing.

According to one embodiment, when the cap is assembled to the syringe housing, the at least one flexible rib may be configured to cause the flexible arm of the distal syringe housing part to move in a radial direction to disengage from the proximally facing surface of the proximal syringe housing part, and the radial protrusion of the at least one flexible arm of the cap engages with the distally facing surface of the proximal syringe housing part in an assembled position of the cap and syringe housing.

According to one embodiment, when the flexible arm of the distal syringe housing part is disengaged from the proximally facing surface of the proximal syringe housing part by the rib, the proximal syringe housing part is movable relative the distal syringe housing part in an axial direction of the syringe housing. Thus, the rib provides for a simple and reliable trigger mechanism for releasing the proximal syringe housing part from the distal syringe housing part.

According to one embodiment, the protrusion of the flexible arm of the distal syringe housing part may comprise a distal facing surface configured to engage with the proximally facing surface of the proximal syringe housing part before the cap is assembled to the syringe housing.

According to one embodiment, the flexible arms of the cap may comprise inclined surfaces that are inclined with respect to the longitudinal axis on a leading distal end of the flexible arms of the cap to interact with a proximally facing inclined surface of the proximal syringe housing part to move the flexible arm radially outwards when the cap is assembled to the syringe housing. The inclined surfaces advantageously facilitate the radial motion of the flexible arms when interacting with the proximally facing inclined surface of the proximal syringe housing part.

According to one embodiment, the rib may comprise an inclined surface with respect to the longitudinal axis on a leading distal end of the cap to interact with a proximally facing inclined surface of the proximal syringe housing part to move the flexible arm of the distal syringe housing part radially outwards to disengage from the proximally facing surface of the proximal syringe housing part when the cap is assembled to the syringe housing. The inclined surfaces advantageously facilitate a gradual radial motion of the flexible arm of the distal syringe housing part.

There is further provided a medicament delivery device comprising the subassembly of any of the herein disclosed embodiments.

Generally, all terms used in the claims are to be interpreted according to their ordinary meaning in the technical field, unless explicitly defined otherwise herein. All references to “a/an/the member, apparatus, component, means, etc.” are to be interpreted openly as referring to at least one instance of the member, apparatus, component, means, etc., unless explicitly stated otherwise.

### BRIEF DESCRIPTION OF THE DRAWINGS

The specific embodiments of the inventive concept will now be described, by way of example, with reference to the accompanying drawings, in which:

FIG. 1 is a perspective view of an autoinjector according to embodiments of the present disclosure;

FIG. 2 is an exploded view of an autoinjector according to embodiments of the present disclosure;

FIG. 3 is an exploded view of an autoinjector according to embodiments of the present disclosure;

FIG. 4A-B are two different views of a distal syringe housing part according to embodiments of the present disclosure;

FIGS. 5A-C are different perspective views of a proximal syringe housing part according to embodiments of the present disclosure;

FIG. 6 illustrates a cap according to embodiments of the present disclosure;

FIG. 7 is a perspective view of a shield member according to embodiments of the present disclosure;

FIG. 8 is a perspective view of a medicament container according to embodiments of the present disclosure;

FIG. 9 is a partial perspective view of the subassembly with the cap assembled to the syringe housing according to embodiments of the present disclosure;

FIG. 10 is a partial perspective view of the subassembly prior to mounting the cap on the syringe housing according to embodiments of the present disclosure;

FIG. 11 is a partial perspective view of the subassembly when the cap is moved closer to the syringe housing according to embodiments of the present disclosure; and

FIG. 12 is a partial perspective view of the subassembly when the cap is moved to the syringe housing according to embodiments of the present disclosure.

### DETAILED DESCRIPTION

The inventive concept will now be described more fully hereinafter with reference to the accompanying drawings, in which exemplifying embodiments are shown. The inventive concept may, however, be embodied in many different forms and should not be construed as limited to the embodiments set forth herein; rather, these embodiments are provided by way of example so that this disclosure will be thorough and complete, and will fully convey the scope of the inventive concept to those skilled in the art. Like numbers refer to like members throughout the description.

FIG. 1 shows an example of a medicament delivery device 1 such as an autoinjector according to embodiments of the present disclosure. The medicament delivery device 1 is configured to expel medicament from a medicament container 16 via a medicament delivery member such as a needle, to a user at a dose delivery site. The medicament delivery device 1 extends from a proximal end 6a to a distal end 6b relative to the axis 102.

The medicament delivery device 1 comprises a housing 3 with a window 4. The housing 3 has a proximal end 3a and a distal end 3b. A cap 7 covers the proximal end 3a of the housing 3.

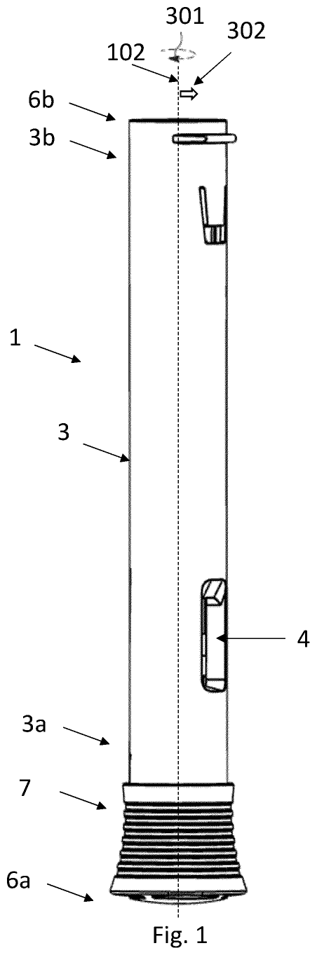

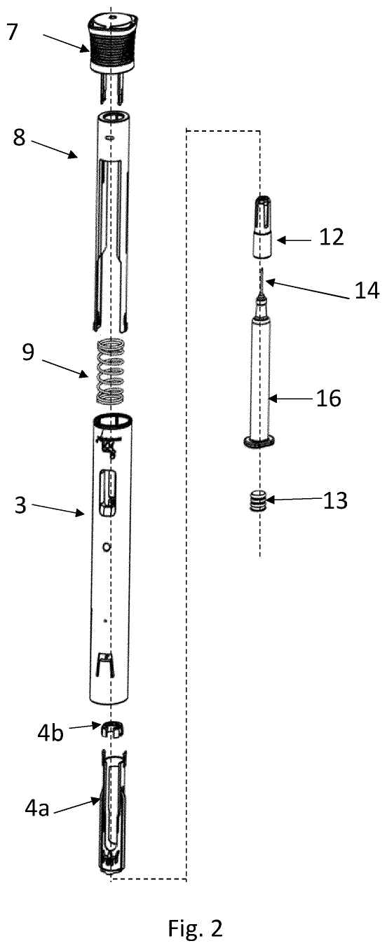

FIG. 2 is an exploded view of the medicament delivery device 1 which further comprises a cover structure 8 arranged in the housing 3 and extends proximally from the proximal end 6a. The cover structure 8 is configured to be moved linearly relative to the housing 3 from an extended position to a retracted position in which the cover structure 8 is received further in the housing 3 and in which a medicament delivery member such as a needle 14 is exposed.

The cover structure 8 is biased in the proximal direction towards the extended position by a spring 9.

A plunger 13 is adapted to move in the medicament container to force a medicament out from the medicament container 16.

The medicament delivery device 1 comprises the subassembly 2 which will now be described in more detail with reference to subsequent drawings.

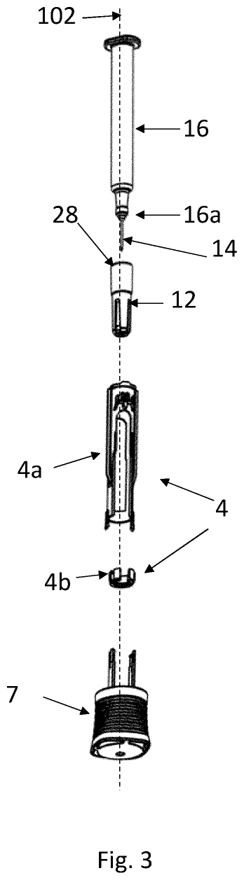

An exploded view of the subassembly is shown in FIG. 3. The subassembly 2 comprises a syringe housing 4 having a proximal housing part 4a and a distal syringe housing part 4b that are separable from each other.

The subassembly 2 further comprises a removable shield member 12 configured to cover the needle 14. The needle 14 is attached to a proximal end 16a of the medicament container 16 accommodated in the syringe housing 4. The needle 14 is adapted to deliver a medicament contained in the medicament container 16 to an injection site. The shield member 14 is a removable needle shield that protects a user from sharp injury prior to use.

The subassembly 2 further comprises a cap 7 configured to cover the shield member 12 on the proximal end 6a of the medicament delivery device 1. The cap 7 is removable from the housing 3.

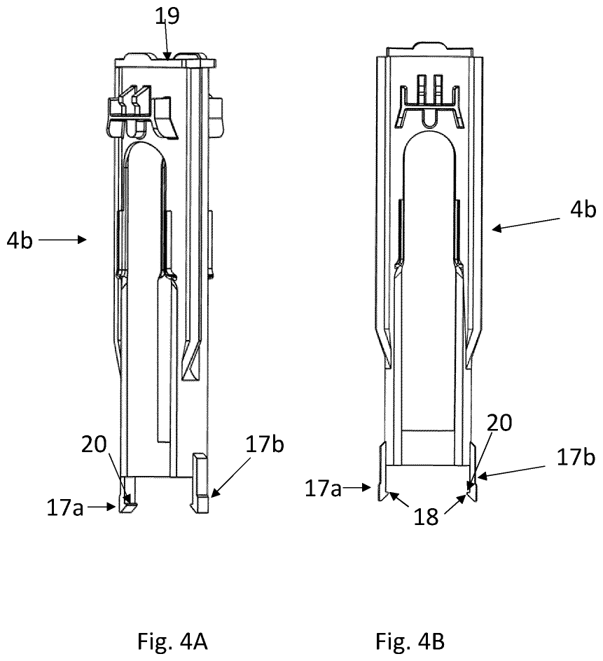

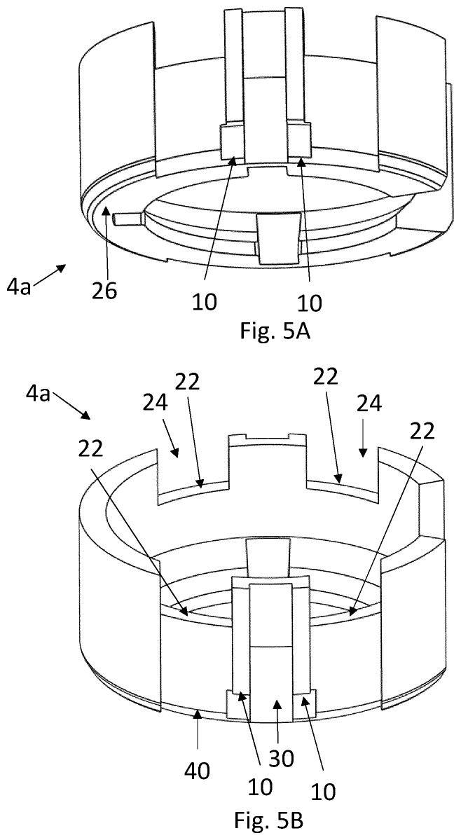

FIGS. 4A-B are two different views of the distal syringe housing part 4b which comprises a flexible arm 17a extending towards the proximal syringe housing part 4a see e.g., FIG. 3. The flexible arm 17a comprises a protrusion 18 configured to engage with a proximally facing surface 10 of the proximal syringe housing part 4a, better seen in FIGS. 5A-C. Further, the protrusion 17a comprises a distally facing surface 20.

In this embodiment, the distal syringe housing part 4b comprises two oppositely arranged flexible arms 17a-b with respect to the longitudinal axis 102. Each of the flexible arms 17a-b comprises a protrusion 18 with a respective distally facing surface 20. The protrusions 18 extend radially inwards.

FIGS. 5A-C illustrate different perspective views of the proximal syringe housing part 4a. The proximal syringe housing part 4a comprises at least one proximally facing surface 10 configured to engage with the distally facing surfaces 20 of the protrusions 17a-b of the distal syringe housing part 4b, prior to attaching the cap 7 to the syringe housing 4. The proximally facing surfaces 10 are the short sides of a ridge 11 or edge of the proximal syringe housing part 4a that reaches along the longitudinal direction 102.

Between the ridges 11, the proximal syringe housing part 4a comprises a proximally facing inclined surface 30. Between the ridges 11 a groove 32 is formed.

The proximal syringe housing part 4a further comprises a set of distally facing surfaces 22. The distally facing surfaces 22 are here exemplified as side walls of a respective cut-out 24 of which two are numbered. The cut-outs 24 may be replaced by grooves or holes in the material of the proximal syringe housing part 4a as long as a distally facing surface 22 is obtained.

On a proximal end of the proximal syringe housing part 4a, the proximal syringe housing part 4a comprises a proximally facing surface 26. The proximally facing surface 26 faces towards the shield member 12 and is here shown as the proximal-most surface of the proximal syringe housing part 4a.

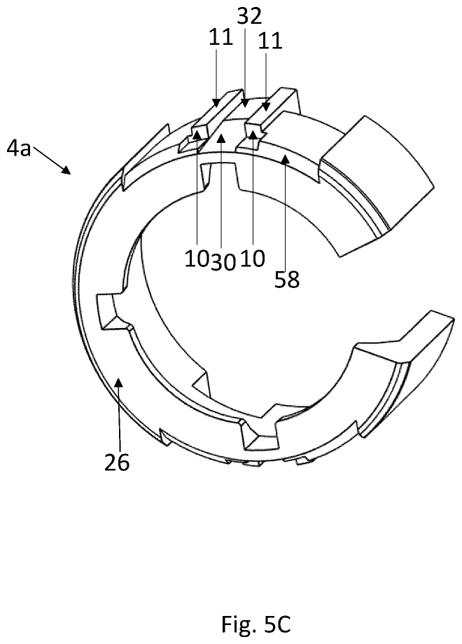

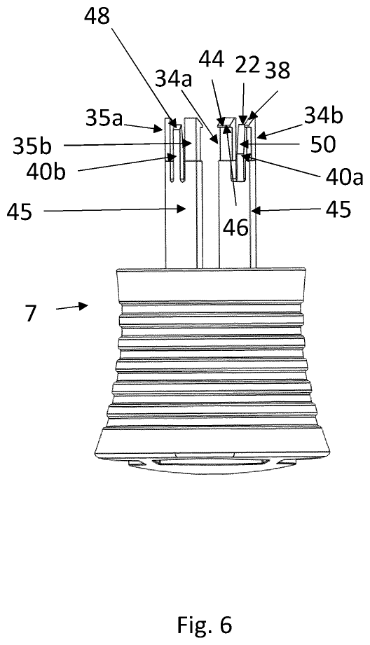

FIG. 6 illustrates a cap 7 that is configured to cover the shield member 12. The cap 7 comprises at least one flexible arm 34a extending distally towards the syringe housing 4. In the illustrated embodiment, the cap 7 comprises two pairs of flexible arms, the first pair comprising a first flexible arm 34a and a second flexible arm 34b. The second pair of flexible arms 35a-b is arranged opposite the first pair of flexible arms 34a-b with respect to the longitudinal axis 102.

The cap 7 further comprises at least one flexible rib 40a. In the illustrated embodiment the cap 7 comprises a first rib 40a and a second rib 40b opposite the first rib 40a with respect to the longitudinal axis. The ribs 40a, and 40b extend in parallel. Further, the ribs 40a and 40b extend in parallel with the flexible arms 34a-b , and 35a-b along the longitudinal axis 102. In addition, the ribs 40a and 40b and the flexible arms 34a-b , and 35a-b extend from a base arm 45 that is attached to the body of the cap 7.

The flexible arms 34a-b and 35a-b of the cap 7 comprises a respective radial protrusion 44 that is configured to engage with the distally facing surface 22 of the proximal syringe housing part 4a. The protrusion 44 extends radially inwards and comprises a proximally facing surface 46 configured to engage with the respective distally facing surface 22 of the proximal syringe housing part 4a.

Further, the flexible arms 34a-b and 35a-b of the cap 7 each comprise inclined surfaces 38 that are inclined with respect to the longitudinal axis 102 on a leading distal end of the flexible arms 34a-b and 35a-b . The inclined surfaces provide for a pointy leading edge of the flexible arms 34a-b and 35a-b.

The ribs 40a and 40b each comprise an inclined surface 48 with respect to the longitudinal axis 102 on a leading distal end of the respective rib 40a and 40b. The inclined surface 48 faces radially outwards and in the distal direction. Further, the ribs 40a and 40b each comprise a further inclined surface 50 being inclined with respect to the longitudinal axis on the leading distal end of the rib 40a-b . The inclined surface 50 faces radially inwards and in the distal direction. The inclined surfaces 48 and 50 are located on opposite side of the respective rib 40a-b in the radial direction. Additionally, the inclined surfaces 48 and 50 meet at the pointy edge of the respective rib 40a-b.

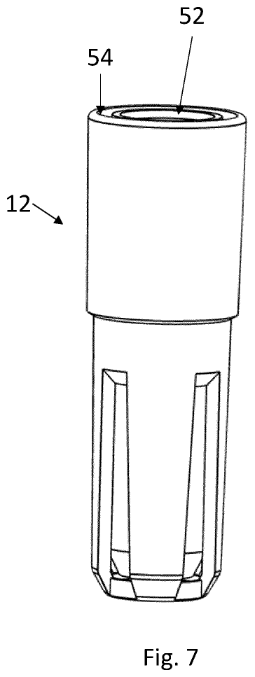

FIG. 7 is a perspective view of a shield member 12, e.g., a removable needle shield. The shield member is overall cylindrical with an inner space 52 for receiving a needle 14. The shield member 12 comprises a distally facing surface 54 configured to engage with the proximally facing surface 26 of the proximal syringe housing part 4a. This engagement can lead to that the shield member 12 moves in the axial direction to expose the needle 14.

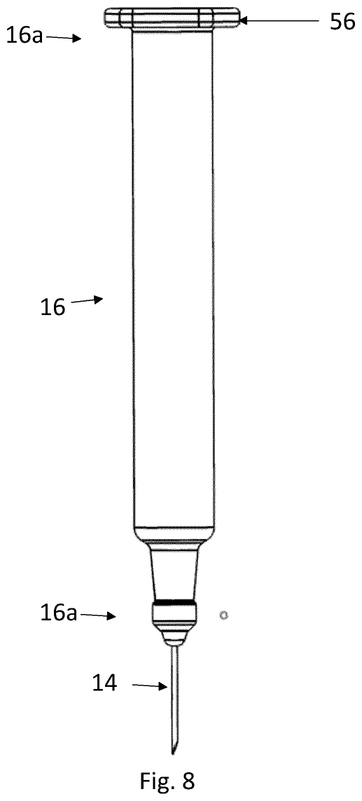

FIG. 8 is a perspective view of a medicament container 16 having thereon an attached needle 14 at its proximal end 16a. Further, the medicament container 16 comprises a radially extending flange 56 on a distal end 16b of the medicament container 16. The flange is adapted to prevent the distal syringe housing part 4b from moving distally. The medicament container 16 is arranged inside the distal syringe housing part 4b.

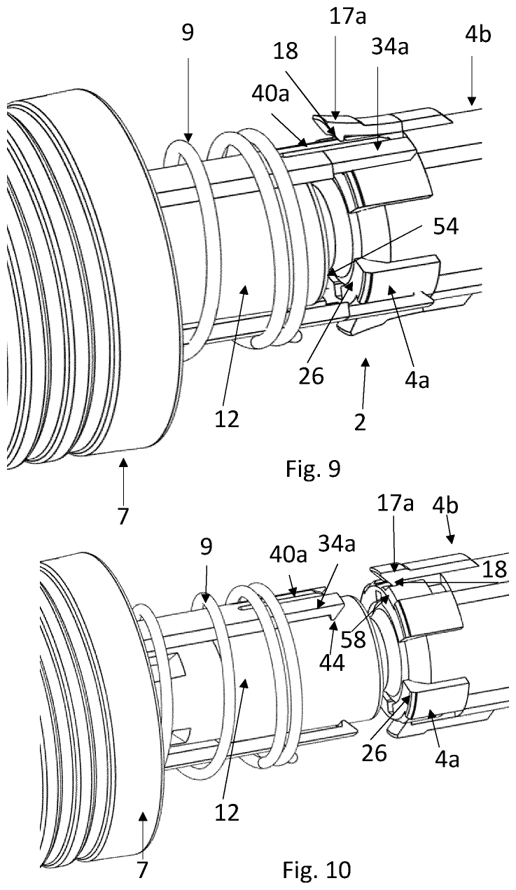

FIG. 9 is a partial perspective view of the subassembly 2 with the cap 7 assembled to the syringe housing 4. In this assembled state, the at least one flexible rib 40a,b is located between the flexible arm 17a,b of the distal syringe housing part 4b and the proximal syringe housing part 4a. In other words, the flexible rib 40a is interleaved between the flexible arm 17a and the proximal syringe housing part 4a. Preferably, the flexible rib 40a is arranged in the groove 32 formed between the ridges 11 described with reference to FIG. 5C. When the rib 40a is located in this position, the flexible arm 17a is flexed radially outwards by the rib 40a which prevents the protrusion 18, which extends radially inwards of the flexible arm 17a to engage with the proximally facing surface 10 of the proximal syringe housing part 4a. In other words, in this position, the proximal syringe housing part 4a is movable along the axis 102 in the proximal direction with respect to the distal housing part 4b.

Further, radial protrusions 44, see FIG. 6 are here in FIG. 9 engaged with the distally facing surfaces 22 of the proximal syringe housing part. This means that when the cap 7 is disassembled from the syringe housing 4 by moving the cap 7 proximally relative the syringe housing 4, the engagement between the radial protrusion 44 of the at least one flexible arm 34a-b , 35a-b of the cap 7 and the distally facing surface 22 of the proximal syringe housing part 4a causes the proximal syringe housing part 4a to move proximally relative the distal syringe housing part 4b. As the cap 7 moves the proximal syringe housing part 4a further proximally by the engagement between the distally facing surface 22 of the proximal syringe housing part 4a and the proximally facing surface 46 of the flexible arms 34a,b , 35a,b , the proximally facing surface 26 of the proximal syringe housing part 4a abuts against the distally facing surface 54 of the shield member 12. The thereby generated force acting on the shield member 12 causes the shield member 12 to move in the axial direction to expose the medicament delivery member 14.

FIG. 10 illustrates the subassembly 2 prior to mounting the cap 7 on the syringe housing 4. Here, the distally facing surface 20 of the protrusion 18 of the flexible arm 17a is engaged with the proximally facing surface 10 of the proximal housing part 4a. This engagement prevents proximal axial motion of the proximal housing part 4a with respect to the distal housing part 4b.

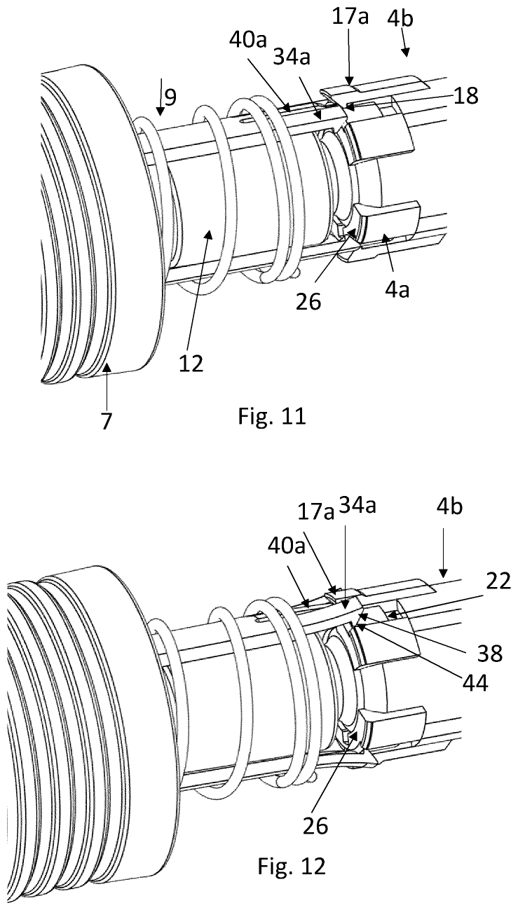

FIG. 11 illustrates the subassembly 2 when the cap 7 is moved closer to the syringe housing 4. The rib 40a is nearly received in the groove 32 of the proximal syringe housing part 4a located radially inside or under the flexible arm 17a. The distally facing surface 20 of the protrusion 18 of the flexible arm 17a is still engaged with the proximally facing surface 10 of the proximal housing part 4a.

In FIG. 12, the cap 7 is moved so that the inclined surface 38 of the protrusion 44 of the flexible arm 34a interacts with a proximally facing inclined surface 58 of the proximal syringe housing part 4a to move the flexible arm 34a radially outwards. Further, the rib 40a is now received in the groove 32. To prevent that the distal syringe housing part 4b is moved distally with respect to the medicament container 16, the distal syringe housing part 4b is configured to abut against the flange 56, best seen in FIG. 8, on a distal end of the medicament container 16.

When the cap 7 is further moved distally to be assembled to the syringe housing, the at least one flexible rib 40a is configured to cause the flexible arm 17a of the distal syringe housing part 4b to move in a radial direction, as is illustrated in FIG. 9, to disengage from the proximally facing surface 10 of the proximal syringe housing part 4a. As discussed with reference to FIG. 9, the radial protrusion 44 of the at least one flexible arm 34a,b , 35a,b of the cap 7 engages with the distally facing surface 22 of the proximal syringe housing part 4a in the final assembled position of the cap 7 and syringe housing 4.

When the rib 40a is received in the grove 32, the inclined surface 50 of the rib interact with the proximally facing inclined surface 40 of the proximal syringe housing part 4a in the groove 32.

This facilitates moving the flexible arm 17a of the distal syringe housing part 4b gradually radially outwards to disengage from the proximally facing surface 10 of the proximal syringe housing part 4b when the cap is assembled to the syringe housing 4.

A medicament delivery device (such as an autoinjector) may generally include various other components. For example, a sensor unit which may recognize injection events, such as the autoinjector inserted into an attachment portion of e.g., a pad, injection started, and injection ends, a memory unit which is configured to store the recorded data during the injection, a connectivity unit configured to transmit the stored data to a smart device or the network directly, a processing unit configured to control the entire system and processes the data before transmitting it, and/or user interface units that are configured to provide feedback to the patient, such as status LEDs, haptic, and/or audio feedback.

When the medicament delivery device is placed into the attachment portion, the sensors inside of the support pad are configured to recognize the event and give feedback to the patient via haptic/visual or audio elements.

When the injection finishes, the sensors are configured to recognize the event and give feedback to the patient again. Further, the collected data is stored in the memory unit and may be transmitted to the smart device/network via the connectivity unit after the injection event finishes.

The sensor can be one of or the combination of the following: a mechanical switch, a Hall-effect sensor, an accelerometer.

The mechanical switch, hall-effect sensor, or accelerometer can be used for detection of the insertion of the auto-injector into an injection port.

The accelerometer can be used for detecting injection events.

Possible wireless communication methods include Bluetooth and Cellular Networks.

Bluetooth connectivity requires a smart device to transmit the stored data to the network and it requires a pairing action between the support pad and the smart device before being able to use the supporting pad. But it's a cheaper alternative and it requires less space on PCB.

The cellular network does not require any pairing process, it can be used as a plug-n-play device, no prior setup is needed, but it's more expensive and it requires more space on PCB.

Depending on the requirements of the product any of those two technologies can be used.

Such processing units may comprise a logic circuit or control unit including a microprocessor, microcontroller, programmable digital signal processor or another programmable device. The processing circuitry may also, or instead, each include an application specific integrated circuit, a programmable gate array or programmable array logic, a programmable logic device, or a digital signal processor. Where the processing circuitry includes a programmable device such as the microprocessor, microcontroller or programmable digital signal processor mentioned above, the processor may further include computer executable code that controls operation of the programmable device.

The medicament delivery devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary types of drugs that could be included in the medicament delivery devices described herein include, but are not limited to, antibodies, proteins, fusion proteins, peptibodies, polypeptides, pegylated proteins, protein fragments, protein analogues, protein variants, protein precursors, and/or protein derivatives. Exemplary drugs that could be included in the medicament delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-1a (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2 diabetes, obesity), dupilumab (atopic dermatitis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)). Pharmaceutical formulations including, but not limited to, any drug described herein are also contemplated for use in the medicament delivery devices described herein, for example pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

The inventive concept has mainly been described above with reference to a few examples. However, as is readily appreciated by a person skilled in the art, other embodiments than the ones disclosed above are equally possible within the scope of the inventive concept, as defined by the appended claims.

1-15 (canceled)

16. A subassembly of a medicament delivery device, the subassembly comprising:
a syringe housing having a proximal syringe housing part and a distal syringe housing part, the distal syringe housing part comprising a flexible arm extending towards the proximal syringe housing part, the flexible arm comprising a protrusion configured to engage with a proximally facing surface of the proximal syringe housing part;
a shield member configured to cover a medicament delivery member, the medicament delivery member being attached to a proximal end of a medicament container accommodated in the syringe housing; and
a cap configured to cover the shield member, the cap comprising at least one flexible arm extending distally towards the syringe housing, the cap further comprising at least one flexible rib located between the flexible arm of the distal syringe housing part and the proximal syringe housing part to prevent engagement between the protrusion of the flexible arm of the distal housing part and the proximally facing surface of the proximal syringe housing part, wherein the at least one flexible arm of the cap comprises a radial protrusion configured to engage with a distally facing surface of the proximal syringe housing part,
wherein when the cap is disassembled from the syringe housing by moving the cap proximally relative the syringe housing, the engagement between the radial protrusion of the at least one flexible arm of the cap and the distally facing surface of the proximal syringe housing part causes the proximal syringe housing part to move proximally relative the distal syringe housing part.

17. The subassembly according to claim 16, the shield member comprising a distally facing surface configured to engage with a proximally facing surface of the proximal syringe housing part, whereby the shield member is configured to move in the axial direction to expose the medicament delivery member.

18. The subassembly according to claim 16, wherein the protrusion of the at least one flexible arm of the cap extends radially inwards and comprises a proximally facing surface configured to engage with the distally facing surface of the proximal syringe housing part.

19. The subassembly according to claim 16, wherein the proximal syringe housing part comprises a groove or hole or cut-out, wherein the distally facing surface is a side wall of the groove or hole or cut-out.

20. The subassembly according to claim 16, wherein the rib is a first rib, and wherein the cap comprises a second rib opposite the first rib with respect to a longitudinal axis of the medicament delivery device.

21. The subassembly according to claim 16, wherein the flexible arm is one of a first pair of flexible arms of the cap, the cap further comprising a second pair of flexible arms opposite the first pair of flexible arms with respect to the longitudinal axis of the medicament delivery device.

22. The subassembly according to claim 16, wherein the protrusion of the flexible arm of the distal syringe housing part extends radially inwards.

23. The subassembly according to claim 16, the proximal syringe housing part comprising a groove radially inside the flexible arm of the distal syringe housing part for receiving the rib.

24. The subassembly according to claim 16, wherein the distal end of the distal syringe housing part is configured to abut against a flange on a distal end of the medicament container.

25. The subassembly according to claim 16, wherein when the cap is assembled to the syringe housing, the at least one flexible rib is configured to cause the flexible arm of the distal syringe housing part to move in a radial direction to disengage from the proximally facing surface of the proximal syringe housing part, and the radial protrusion of the at least one flexible arm of the cap engages with the distally facing surface of the proximal syringe housing part in an assembled position of the cap and syringe housing.

26. The subassembly according to claim 25, wherein, when the flexible arm of the distal syringe housing part is disengaged from the proximally facing surface of the proximal syringe housing part by the rib, the proximal syringe housing part is movable relative the distal syringe housing part in an axial direction of the syringe housing.

27. The subassembly according to claim 25, wherein the protrusion of the flexible arm of the distal syringe housing part comprises a distal facing surface configured to engage with the proximally facing surface of the proximal syringe housing part before the cap is assembled to the syringe housing.

28. The subassembly according to claim 25, wherein the flexible arms of the cap comprise inclined surfaces that are inclined with respect to the longitudinal axis on a leading distal end of the flexible arms of the cap to interact with a proximally facing inclined surface of the proximal syringe housing part to move the flexible arm radially outwards when the cap is assembled to the syringe housing.

29. The subassembly according to claim 25, wherein the rib comprises an inclined surface with respect to the longitudinal axis on a leading distal end of the cap to interact with a proximally facing inclined surface of the proximal syringe housing part to move the flexible arm of the distal syringe housing part radially outwards to disengage from the proximally facing surface of the proximal syringe housing part when the cap is assembled to the syringe housing.

30. A medicament delivery device comprising the subassembly according to claim 16.
